(19) United States
(12) Patent Application Publication
(10) Pub. No.: US 2025/0186338 A1
(43) Pub. Date: Jun. 12, 2025

(54) Stable Soft Chews, and Methods to Produce Same

(71) Applicant: Florida Research Group, LLC, Tampa, FL (US)

(72) Inventor: Kotha SEKHARAM, Tampa, FL (US)

(21) Appl. No.: 18/969,629

(22) Filed: Dec. 5, 2024

Related U.S. Application Data
(60) Provisional application No. 63/607,369, filed on Dec. 7, 2023.

(51) Int. Cl. A61K 9/00; A61K 31/7008; A61K 31/737; A61K 47/02; A61K 47/12; A61K 47/44

(52) U.S. Cl. CPC A61K 9/0056; A61K 31/7008; A61K 31/737; A61K 47/02; A61K 47/12; A61K 47/44

(57) ABSTRACT

The subject invention provides methods to produce stable soft chews comprising joint-health-promoting ingredients. The subject invention further provides novel, stable soft chews having joint-health-promoting ingredients.

### CROSS REFERENCE TO A RELATED APPLICATION

This application claims the benefit of U.S. Provisional Application Ser. No. 63/607,369, filed Dec. 7, 2023, which is incorporated herein by reference in its entirety.

### BACKGROUND OF THE INVENTION

Functional health ingredients are delivered to end users in a variety of different ways. One very common form is a solid dosage. Tablets, chewable tablets, orally disintegrating tablets, capsules, and powders are some examples of solid oral dosages. Soft chews are gaining more attention because of a preference for their bite, texture, chewiness, flavor and overall mouthfeel.

Soft chews are generally made by combining active ingredients with excipients, binders, humectants, oils, sweeteners, flavors, colors and preservatives to make a dough and then shaping the dough to desired shape, size and weight.

If the composition is not appropriate, soft chews become hard and fail to perform their intended purpose. Also, if the composition is not proper, it may affect the stability of certain active ingredients. For example, the presence of water may affect moisture-sensitive functional ingredients. Further, if the process temperature is high, it may degrade thermo-labile active ingredients; if the composition includes oxidation-triggering ingredients, it may affect the stability of oxidation-prone active ingredients; and high water activity may affect enzyme activity as well as viability of probiotics.

Having so many factors affecting finished forms, there is always a need for improved compositions and methods to make soft chews.

Edwin Christensen (WO 2006/036552 A2) reported making a nutritional soft chew product with 10% to about 50% by weight of a starch component, about 25% to about 50% by weight of a sugar component, about 0-20% of a humectant component, and about 5% to about 25% by weight of water.

U.S. Pat. No. 8,114,455 B2 (Neil E. Paulsen et al.) teaches a process for manufacturing chewable dosage forms for drug delivery with an active ingredient, an inactive ingredient, and non-aqueous fluid, wherein the non-aqueous fluid is an oil such as corn, safflower, cottonseed, soybean and olive oils.

U.S. Pat. No. 8,865,240 B2 (Neil E. Paulsen et al.) teaches a process for manufacturing an edible soft chewable medication comprising blending an active ingredient with an oil and dry inactive ingredients including a flavoring, a binder, a bulking agent, a humectant and an excipient.

Joint-health-promoting supplements are typically formulated with active ingredients including glucosamine hydrochloride, glucosamine sulfate, N-acetyl glucosamine, and/or chondroitin sulfate, and delivered as, for example, tablets, capsules, oral liquids or soft chews.

Ingredients such as glucosamine are prone to chemical degradation due to moisture, temperature, and interaction with inactive ingredients of the formulations. Usage of antioxidants to stabilize glucosamine has not proven to be fully effective.

U.S. Pat. No. 4,642,340 teaches methods to stabilize glucosamine sulfate by forming a crystalline mixed salt of glucosamine sulphate with an alkali halide (sodium chloride). The mixed salt of glucosamine and sodium chloride was prepared dissolving both components at a certain proportion and temperature, precipitating by using a solvent, and recovering the salt and drying. However, for safety and environmental considerations, usage of a solvent is undesirable.

EP-214642 describes an improved method for formation of a mixed salt of glucosamine sulphate with alkali halide, specifically, in this case, with potassium chloride.

EP-444000 describes the stabilization of an oral dosage form of glucosamine sulphate by providing ascorbic acid as an anti-oxidant in an amount being at least 4 of that of glucosamine sulphate. Calcium carbonate was used as a desiccant. The formulation is suited for manufacturing oral dosage forms such as tablets, most preferably capsules.

U.S. Pat. No. 9,592,202 teaches a method to stabilize glucosamine by (a) granulating glucosamine with a disintegrant and a binder; (b) a calcium compound is granulated separately with a disintegrant and a binder; and (c) mixing the above (a) with (b), and other active or inactive ingredients, to form tablet or caplet.

Two independent granulation steps, followed by blending and tableting adds cost and complexity to the process. In addition, this and all the above methods describe usage of glucosamine in tablets or capsules, but not in chews.

For use in companion animals, soft chews are the most preferred dosage form, compared to tablets and capsules.

As noted above, soft chews are typically made by blending powdered active ingredients and inactive ingredients with added water or by adding water-containing syrups, kneading to make a dough, and the dough is extruded to form a chew of desired shape, size and weight.

Unfortunately, having a substantial amount of added water in soft chew formulation accelerates degradation of glucosamine.

Hence, there is a need to develop an improved manufacturing method to prepare stable soft chews with joint-health-promoting ingredients by a simple, less expensive process and without a need for any granulation steps.

### BRIEF SUMMARY OF THE INVENTION

The subject invention provides methods to stabilize soft chews comprising joint-health-promoting ingredients. The subject invention further provides novel, stable soft chews having joint-health-promoting ingredients.

In a preferred embodiment, the subject invention provides a method of manufacturing a stable joint-health-promoting soft chew, wherein the method comprises:
a) blending a joint-health-promoting ingredient with an edible grade inorganic salt to form Mix-1;
b) blending a long chain fatty acid with 13 to 21 carbon atoms with a solid fat, and melting them together at a temperature not less than 70° C. to form Mix-2;
c) spraying Mix-2 onto Mix-1 while blending them to form a uniform Mix-3;
d) cooling Mix-3 to room temperature;
e) blending Mix-3 with other active ingredients and/or excipients to form a shapeable dough;
f) extruding the shapeable dough formed in e) to make a chew of desired shape and weight; and
g) curing the chew at room temperature for about 24 hours;
or
d′) blending inactive ingredients through a cooker-extruder to form a continuous hollow tube to form Mix-4;
e′) filling the continuous hollow tube with a desired amount of Mix-3 through a second extruder, cutting to a desired length, and
f′) curing the chew at room temperature.

In preferred embodiments, the joint-health-promoting ingredient is selected from glucosamine hydrochloride, glucosamine sulfate, N-acetyl glucosamine, and chondroitin sulfate.

The excipient can be selected from, for example, starch, cellulose, flour, sorbitol, sucrose, mannitol, other sugars, brewer's dried yeast, calcium carbonate, dicalcium phosphate, or a combination thereof. In a preferred embodiment, the excipient is starch. The excipient may comprise, for example, 5% to 75%, 10% to 60%, 20% to 50%, or 30% to 40% of the soft chew.

In preferred embodiments, the edible grade inorganic salt is selected from calcium carbonate, dicalcium phosphate, tricalcium phosphate, calcium gluconate, calcium lactate, calcium citrate, calcium chloride, sodium chloride, sodium bicarbonate, sodium sulfate, potassium chloride, and magnesium chloride.

In a specific embodiment, the edible grade inorganic salt is calcium carbonate.

In preferred embodiments, the long chain fatty acid with 13 to 21 carbon atoms is selected from palmitic acid, stearic acid, arachidic acid, potassium stearate, calcium stearate, magnesium stearate, sodium stearate, zinc stearate, glycol stearate, and stearin.

In a specific embodiment, the long chain fatty acid with 13 to 21 carbon atoms is stearic acid.

In preferred embodiments, the solid fat is selected from beef fat, pork fat, chicken fat, coconut fat, palm fat, palm kernel oil fat, hydrogenated fats, hydrogenated vegetable shortening, and emulsified vegetable shortening.

In preferred embodiments, the ratio of joint-health-promoting ingredient to edible grade inorganic salt is from 50:50 to 80:20.

In preferred embodiments, the ratio of fatty acid with 13 to 21 carbon atoms to solid fat is from 30:70 to 70:30.

The subject invention further provides soft chews made by the methods described herein.

### DETAILED DESCRIPTION OF THE INVENTION

The subject invention provides methods to produce stable soft chews comprising joint-health-promoting ingredients. The subject invention further provides novel, stable soft chews having joint-health-promoting ingredients.

In one embodiment, the subject invention provides a method of manufacturing a stable joint-health-promoting soft chew wherein the method comprises:
a) blending a joint-health-promoting ingredient with an edible grade inorganic salt to form Mix-1;
b) blending a long chain fatty acid with 13 to 21 carbon atoms with a solid fat, and melting them together at a temperature not less than 70° C. to form Mix-2;
c) spraying Mix-2 onto Mix-1 while blending them to form a uniform Mix-3;
d) cooling Mix-3 to room temperature;
e) blending Mix-3 with other active ingredients and/or excipients to form a shapeable dough;
f) extruding the shapeable dough formed in e) to make a chew of desired shape and weight; and
g) curing the chew at room temperature for, for example, about 24 hours.

In another embodiment, the subject invention provides a method of manufacturing a stable joint-health-promoting soft chew wherein the method comprises:
a) blending a joint-health-promoting ingredient with an edible grade inorganic salt to form Mix-1;
b) blending a long chain fatty acid with 13 to 21 carbon atoms with a solid fat, and melting them together at a temperature not less than 70° C. to form Mix-2;
c) spraying Mix-2 onto Mix-1 while blending them to form a uniform Mix-3;
d) blending inactive ingredients through a cooker-extruder to form a continuous hollow tube to form Mix-4; and
e) filling the continuous hollow tube with a desired amount of Mix-3 through a second extruder, cutting to a desired length, and
f) curing the chew at room temperature for, for example, about 24 hours.

The blending can be done in, for example, a blender.

In preferred embodiments, the joint-health-promoting ingredient is selected from glucosamine hydrochloride, glucosamine sulfate, N-acetyl glucosamine, and chondroitin sulfate.

The excipient can be selected from, for example, starch, cellulose, flour, sorbitol, sucrose, mannitol, other sugars, brewer's dried yeast, calcium carbonate, dicalcium phosphate, or a combination thereof. In a preferred embodiment, the excipient is starch. The excipient may comprise, for example, 5% to 75%, 10% to 60%, 20% to 50%, or 30% to 40% of the edible soft chew.

In preferred embodiments, the edible grade inorganic salt is selected from calcium carbonate, dicalcium phosphate, tricalcium phosphate, calcium gluconate, calcium lactate, calcium citrate, calcium chloride, sodium chloride, sodium bicarbonate, sodium sulfate, potassium chloride, and magnesium chloride.

In a specific embodiment, the edible grade inorganic salt is calcium carbonate.

In preferred embodiments, the long chain fatty acid with 13 to 21 carbon atoms is selected from palmitic acid, stearic acid, arachidic acid, potassium stearate, calcium stearate, magnesium stearate, sodium stearate, zinc stearate, glycol stearate, and stearin.

In a specific embodiment, the long chain fatty acid with 13 to 21 carbon atoms is stearic acid.

In preferred embodiments, the ratio of joint-health-promoting ingredient to edible grade inorganic salt is from 50:50 to 80:20, including, for example, 75:25, and all ratios therebetween.

In preferred embodiments, the ratio of fatty acid with 13 to 21 carbon atoms to solid fat is from 30:70 to 70:30, including, for example, 40:60, 50:50, 60:40, and all ratios therebetween.

The solid fat can be selected from, for example, vegetable shortening, beef tallow, pork fat, stearic acid, magnesium stearate, and combinations thereof. Preferably, the solid fat comprises fat that remains solid at 20° C. and has a melting point of 30° C. or above. In a preferred embodiment, the fat constitutes from about 1 to about 15% (w/w) of the soft chew.

The additional active ingredient can be selected from, for example, anthelmetics, analgesics, antibiotics, antivirals, anti-inflammatories, endoparasiticides, ectoparasiticides, antifungals, sedatives, anxiolytics, health supplements, therapeutic agents, analgesics, antacids, antianxiety drugs, antiarrhythmics, antibacterials, antibiotics, anticoagulants, anticonvulsants, antidepressants, antidiarrheals, antiemetics, antifungals, antihistamines, antihypertensives, anti-inflammatories, antineoplastics, antipsychotics, antipyretics, antivirals, barbiturates, beta-blockers, bronchodilators, cold cures, corticosteroids, cough suppressants, cytotoxics, decongestants, diuretics, expectorants, hormones, hypoglycemics (oral), immunosuppressives, laxatives, muscle relaxants, sedatives, sex hormones, sleep inducers, tranquilizers, vitamins, and combinations thereof.

Other active agents include: non-steroidal anti-inflammatory drugs (e.g, carprofen, flunixine meglumine, ketoprofen, ketoprofen methyl ester, naproxen, meloxicam, robenacoxib, and firocoxib), medetomidine, phenylbutazone, hydromorphone, anti-emetics (e.g., maropitant, and maropitant salts, dextromethorphan, diphenhydramine, 8-chlorotheophylline, cisapride, omeprazol, famotidine, metoclopramide, promethazine, dolasetron, ondansetron, granisetron, ketamine, lansoprasol, meclizine, and mirtazepine), antihistamines/antipyretics (e.g., acepromazine, clemastine fumarate, cyproheptadine, famotidine, loratadine, hydroxyzine, meclizine hydrochloride, apoquel, chlorpheniramine, and diphenhydramine), antiparasitics (e.g., macrocyclic lactones (ivermectin, abamectin, doramectin, emamectin, moxidectin, milbemycin, and milbemycin oxime), imidacloprid, emodepside, levamisole, pyrantel, pyrantel pamoate, isoxazolines (e.g., sarolaner, afoxolaner, lotilaner, and fluralaner), derquantel, anticoccidials, benzimidazoles (thiabendazole, mebendazole, fenbendazole, oxfendazole, and albendazole), antimicrobials (e.g., pleuromutilins, polymyxins, aminoglycosides, fluoroquinolones (e.g., danofloxacin, ciprofloxacin, norfloxacin, ofloxacin, and levofloxacin), macrolides (e.g., azithromycin, erythromycin, and telithromycin), lincosamides (e.g., clindamycin), aminoglycosides (e.g., amikacin, streptomycin, and tobramycin), sulfonamides (e.g., sulfadoxine, sulfamethizole, and sulfisoxazole), penicillins, beta-lactams, tetracyclines (e.g., doxycycline hyclate, and minocycline), aminopenicillins, cephalosporins (1^{st}-4th generations, e.g., simplicef, ceftiofur, and cefovecin), prednisolone, and methylprednisolone.

Combinations of one or more active agents are also envisioned. Additional active agents are known to a person of ordinary skill in the art and such embodiments are within the purview of the invention.

Further optional ingredients that can be used in the process of the subject invention include, for example, probiotics, flavorings, colorings, palatants, and/or sweeteners.

Preferred probiotics include: Lactobacillus acidophilus, Bifidobacterium thermophilum, B. longum, L fermentum, L casei, B. bifidum, E. faecium, and B. coagulans.

Flavoring agents such as molasses, carrot, apple, bacon, hickory flavor; coloring agents such as iron oxide, and titanium dioxide; sweeteners such as sugar, dextrose, sodium saccharin, and sucralose; preservatives such as parabens, propionate, and sorbic acid; and antioxidants such as hydroxyanisole (BHA), butylated hydroxytoluene (BHT), and tertiary-butyl hydroquinone (TBHQ) can also be added. These ingredients can be added in any of the several mixing steps in the methods of the invention described above.

The subject invention further provides stable soft chews. The stable soft chews of the subject invention include those that are made by a process described above. In preferred embodiments, the stable soft chews of the subject invention comprise a joint-health-promoting ingredient as well as an edible grade inorganic salt, a long chain fatty acid with 13 to 21 carbon atoms, and a solid fat. Optionally, the stable soft chew can also comprise other active ingredients and/or excipients.

In preferred embodiments, the joint-health-promoting ingredient is selected from glucosamine hydrochloride, glucosamine sulfate, N-acetyl glucosamine, and chondroitin sulfate.

The excipient can be selected from, for example, starch, cellulose, flour, sorbitol, sucrose, mannitol, other sugars, brewer's dried yeast, calcium carbonate, dicalcium phosphate, or a combination thereof. In a preferred embodiment, the excipient is starch. The excipient may comprise, for example, 5% to 75%, 10% to 60%, 20% to 50%, or 30% to 40% of the edible soft chew.

In preferred embodiments, the edible grade inorganic salt is selected from calcium carbonate, dicalcium phosphate, tricalcium phosphate, calcium gluconate, calcium lactate, calcium citrate, calcium chloride, sodium chloride, sodium bicarbonate, sodium sulfate, potassium chloride, and magnesium chloride.

In a specific embodiment, the edible grade inorganic salt is calcium carbonate.

In preferred embodiments, the long chain fatty acid with 13 to 21 carbon atoms is selected from palmitic acid, stearic acid, arachidic acid, potassium stearate, calcium stearate, magnesium stearate, sodium stearate, zinc stearate, glycol stearate, and stearin.

In a specific embodiment, the long chain fatty acid with 13 to 21 carbon atoms is stearic acid.

In preferred embodiments, the ratio of joint-health-promoting ingredient to edible grade inorganic salt is from 50:50 to 80:20, including, for example, 75:25, and all ratios therebetween.

In preferred embodiments, the ratio of fatty acid with 13 to 21 carbon atoms to solid fat is from 30:70 to 70:30, including, for example, 40:60, 50:50, 60:40, and all ratios therebetween.

The solid fat can be selected from, for example, vegetable shortening, beef tallow, pork fat, stearic acid, magnesium stearate, and combinations thereof. Preferably, the solid fat comprises fat that remains solid at 20° C. and has a melting point of 30° C. or above. In a preferred embodiment, the fat constitutes from about 1 to about 15% (w/w) of the soft chew.

The additional active agents can be selected from, for example, those that are discussed above.

The amount of an active agent included in the soft chews produced according to the methods of the invention ranges from about 0.1% to about 25% w/w of the soft chew. A therapeutically effective amount of an active agent may vary depending upon the intended application, the subject, and the condition being treated, the weight and age of the subject, and the severity of the condition. These parameters can readily be determined by one of ordinary skill in the art.

Further optional ingredients that can be present in the soft chews of the subject invention include, for example, probiotics, flavorings, colorings, palatants, and/or sweeteners.

Preferred probiotics include: Lactobacillus acidophilus, Bifidobacterium thermophilum, B. longum, L fermentum, L casei, B. bifidum, E. faecium, and B. coagulans.

Flavoring agents such as molasses, carrot, apple, bacon, hickory flavor; coloring agents such as iron oxide, and titanium dioxide; sweeteners such as sugar, dextrose, sodium saccharin, and sucralose; preservatives such as parabens, propionate, and sorbic acid; and antioxidants such as hydroxyanisole (BHA), butylated hydroxytoluene (BHT), and tertiary-butyl hydroquinone (TBHQ) can also be added.

The subject invention further provides methods of promoting joint health by administering to a subject a stable soft chew of the subject invention.

The subject can be a human or a non-human animal, preferably, a mammal. When the subject is a human, the subject can be, for example, a child or a senior. When the subject is a non-human animal, the subject can be, for example, a domestic pet, such as a dog or a cat. The subject can also be, for example, a horse, cow, pig, camel, or ferret.

### Definitions

As used herein, the singular forms “a”, “an” and “the” are intended to include the plural forms as well, unless the context clearly indicates otherwise. Further, to the extent that the terms “including”, “includes”, “having”, “has”, “with”, or variants thereof are used in either the detailed description and/or the claims, such terms are intended to be inclusive in a manner similar to the term “comprising.”

The transitional term “comprising,” which is synonymous with “including,” or “containing,” is inclusive or open-ended and does not exclude additional, un-recited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention. Use of the term “comprising” contemplates other embodiments that “consist” or “consist essentially of” the recited component(s).

The term “about” means within an acceptable error range for the particular value as determined by one of ordinary skill in the art, which will depend in part on how the value is measured or determined, i.e., the limitations of the measurement system. Where particular values are described in the application and claims, unless otherwise stated the term “about” meaning within an acceptable error range for the particular value should be assumed. In the context of compositions containing amounts of ingredients where the term “about” is used, these compositions contain the stated amount of the ingredient with a variation (error range) of 0-10% around the value (X±10%). Where the term “about” is used to describe target temperatures or durations of time used in certain processes, the target temperatures of durations of time can be varied within a range 0-10% around the target value (X±10%).

The term “w/w” as used herein to describe an amount of an ingredient in the soft chews of the subject invention indicates that the corresponding ingredient is present in the specified weight ratio of the ingredient to the total weight of the soft chew.

In the present disclosure, ranges are stated in shorthand, to avoid having to set out at length and describe each and every value within the range. Any appropriate value within the range can be selected, where appropriate, as the upper value, lower value, or the terminus of the range. For example, a range of 0.1-1.0 represents the terminal values of 0.1 and 1.0, as well as the intermediate values of 0.2, 0.3, 0.4, 0.5, 0.6, 0.7, 0.8, 0.9, and all intermediate ranges encompassed within 0.1-1.0, such as 0.2-0.5, 0.2-0.8, and 0.7-1.0.

When ranges are used herein, such as for dose ranges, combinations and sub-combinations of ranges (e.g., subranges within the disclosed range), specific embodiments therein are intended to be explicitly included.

A “soft chew” as used herein refers to a pharmaceutical unit dose that is solid at room temperature and has a rubbery texture appropriate for mastication in the mouth. Soft chew is particularly suitable for administration of an active agent to a non-human animal, such as pets, because non-human animals do not prefer swallowing pills. Indeed, administering a solid pill to a non-human animal is difficult because of the resistance the non-human animal may provide to such administration. Non-human animals prefer eating a soft chew because the non-human animals can experience and enjoy flavor that can be incorporated in the soft chew. Such soft chews have a softness that is similar to a cooked ground meat patty.

I claim:

1. A method of manufacturing a stable joint-health-promoting soft chew comprising:
a) blending a joint-health-promoting ingredient with an edible grade inorganic salt to form Mix-1;
b) blending a long chain fatty acid with 13 to 21 carbon atoms with a solid fat, and melting them together at a temperature not less than 70° C. to form Mix-2;
c) spraying Mix-2 onto Mix-1 while blending them to form a uniform Mix-3;
d) cooling Mix-3 to room temperature;
e) blending Mix-3 with other active ingredients and/or excipients to form a shapeable dough;
f) extruding the shapeable dough formed in e) to make a chew of desired shape and weight; and
g) curing the chew at room temperature for about 24 hours;
or
d′) blending inactive ingredients through a cooker-extruder to form a continuous hollow tube to form Mix-4;
e′) filling the continuous hollow tube with a desired amount of Mix-3 through a second extruder, cutting to a desired length, and
f′) curing the chew at room temperature for about 24 hours.

2. The method according to claim 1, wherein the edible grade inorganic salt is selected from calcium carbonate, dicalcium phosphate, tricalcium phosphate, calcium gluconate, calcium lactate, calcium citrate, calcium chloride, sodium chloride, sodium bicarbonate, sodium sulfate, potassium chloride, and magnesium chloride.

3. The method according to claim 2, wherein the edible grade inorganic salt is calcium carbonate.

4. The method according to claim 1, wherein the long chain fatty acid with 13 to 21 carbon atoms is selected from palmitic acid, stearic acid, arachidic acid, potassium stearate, calcium stearate, magnesium stearate, sodium stearate, zinc stearate, glycol stearate, and stearin.

5. The method according to claim 4, wherein the long chain fatty acid with 13 to 21 carbon atoms is stearic acid.

6. The method according to claim 1, wherein the solid fat is selected from beef fat, pork fat, chicken fat, coconut fat, palm fat, palm kernel oil fat, hydrogenated fats, hydrogenated vegetable shortening, and emulsified vegetable shortening.

7. The method according to claim 1, wherein the ratio of joint-health ingredient to edible grade inorganic salt is from 50:50 to 80:20.

8. The method according to claim 1, wherein the ratio of fatty acid with 13 to 21 carbon atoms to solid fat is from 30:70 to 70:30.

9. The method according to claim 1, comprising the use of a joint-health-ingredient selected from glucosamine hydrochloride, glucosamine sulfate, N-acetyl glucosamine, and chondroitin sulfate.

10. A stable joint-health-promoting soft chew produced by the method of claim 1.

11. A method for promoting joint health comprising administering to a subject in need of joint health promotion a stable soft chew according to claim 10.
